(19) United States
(12) Patent Application Publication
(10) Pub. No.: US 2022/0000713 A1
(43) Pub. Date: Jan. 6, 2022

(54) NEEDLE WARMING SYSTEM

(71) Applicant: Wellness Allied Inc, Natick, MA (US)

(72) Inventors: Weihui Li, Natick, MA (US); Tao ZHANG, Natick, MA (US)

(21) Appl. No.: 16/917,998

(22) Filed: Jul. 1, 2020

(51) Int. Cl. A61H 39/06; A61H 39/08

(52) U.S. Cl. CPC A61H 39/06; A61H 39/083; A61H 39/086; A61H 2201/0207; A61H 2201/0176; A61H 2201/5043; A61H 2201/0157; A61H 2201/169; A61H 2201/5097

(57) ABSTRACT

A needle warming system includes: a heating tube for heating acupuncture needles; a ring-shaped rechargeable battery for supplying power to the heating tube, and a empty space for fixing the heating tube for charging The battery accepts the charging of the wireless charging unit in a wireless charging mode; a display unit is used to display the working status and charging status of the needle warming system; the control unit is used to control the temperature of the heating tube, and the charging of the rechargeable battery, and display unit. The wireless rechargeable battery avoids the external power supply and the traditional power wiring obstacles; the creation of empty space in the center with ring-shaped rechargeable batteries and heating pads, medicine pads, etc., allows acupuncture needles to enter the heating tube, which is convenient for the implementation of acupuncture warming needles technique. Moreover, the whole needle warming system is compact, small and portable.

### TECHNICAL FIELD

The present invention generally relates to acupuncture assistance techniques, and more specifically, a system to warm needles during acupuncture treatment.

### BACKGROUND

Moxibustion in traditional Chinese medicine is the use of moxibustion material made from moxa leaves to stimulate acupuncture points or specific parts on the body surface, thus to regulate the physiological and biochemical functions of the human body.

Needle warming technique is a method of combining acupuncture with traditional moxibustion. In the process of needle warming treatment, the needle is inserted in the body first. Then the moxa is wrapped around the needle handle and ignited. The heat from burning of the moxa leaves warms up the needle, which in turn conduct the heat to the body tissue.

With the advancement of science and technology, people are constantly making improvements to the warm needle techniques.

In order to eliminate the smoke generated by moxibustion, patent CN2036831U proposed an electronic needle warming system as early as 1989. Based on traditional needle warming technique, the patent adopted the PTC ceramic as the heating material, with the temperature controlled by components like thyristor, transformer and resistance voltage division circuit.

Patent CN10353635B uses polyimide as the metal electric heating film or carbon fiber as the heating body.

Patent CN109303691A and patent CN207928493U use graphene for system heating.

The problems with above warm needle techniques are: size is too big, cost is high, or it is inconvenient to use with power cord being around, especially if multiple units being used simultaneously.

### SUMMARY

Current needle warming techniques all have above mentioned drawbacks, such as large size, high cost, or tangled wires with external power supply being required.

This present patent solves the power cord problem for the needle warming techniques by using a rechargeable battery as a heating power source.

Further, the present patent solves the large size problem by adopting a specially designed ring-shaped rechargeable battery pack, allowing the needle inserted through.

Preferably, the heating tube adopts MCH heating ceramics, which is cheap, efficient in heat generation and is small in volume.

In addition, in order to facilitate charging, the system can use wireless charging system, which is more convenient to use.

According to one aspect of the present invention, a needle warming system is provided, including: a heating tube for warming the acupuncture needles when the acupuncture needle is sheathed in; a ring-shaped rechargeable battery for providing energy to the heating tube and also providing structural support for the heating tube; a wireless charging unit to charge the ring-shaped rechargeable battery in a wireless charging mode; a display unit to display the system working status, battery charging status, battery level and the temperature control status; a control unit to control the temperature of the heating tube, the heating pad, the charging of the rechargeable battery, and the display of the display unit.

Optionally, the needle warming system may further include: a heating pad, located at the bottom of the needle warming system, for directly heating the human skin.

Optionally, the needle warming system may further include: a disposable double-sided adhesive tape, one side adheres to the needle warming system, and the other side adheres to the user's skin, which can prevent cross infections when the needle warming system is used on different patients directly.

Optionally, the double-sided adhesive tape consists a first adhesive layer, a medicine pad and a second adhesive layer. The first adhesive layer is used to adhere the medicine pad to the needle warming system, and the second adhesive layer is used to attach the medicine pad to the skin, and the adhesive layer between the medicine pad and the skin is permeable to the medicine.

Optionally, the needle warming system may further include: heat insulation layers, assembled around the heating tube, and above the heating pad, to contain the heat and save energy, to prevent possible burns from direct contact with the patient's skin or other nearby persons' skin, to prevent the overheating of batteries and electronic components, and to reduce the heat conduction between the heating tube and the heating pad to allow them being controlled separately.

Optionally, the needle warming system may further include: a wireless charging receiving unit, being a ring-shaped coil, assembled at the bottom of the rechargeable battery. When a needle warming system is placed on a charging station, the wireless charging receiving unit receives electricity from the wireless charging transmitter to charge the rechargeable battery.

Optionally, the heating tube may be an MCH ceramic heating element, a PTC ceramic heating element, a polyimide heating film, a carbon fiber heating material or a graphene heating material.

Optionally, the heating tube is configured to connect with a conical guiding tube, therefore, the acupuncture needle can be easily guided into the heating tube when the needle warming system sheaths the acupuncture needle into the heating tube.

Optionally, the needle warming system may further include a normally closed temperature control switch. When the temperature of the heating tube exceeds a certain threshold, the control unit controls the normally closed temperature control switch to automatically cut off the heating circuit.

Optionally, the control unit includes a wireless communication unit to receive remote control from other systems to turn on or off the needle warming system, or to adjust the temperature of the heating tube and heating pad. This can make use of external big data and/or artificial intelligence for more efficient needle warming treatment.

Optionally, the control unit measures the temperature of the heating tube or acupuncture needle through a temperature measuring element. When this temperature exceeds a certain threshold, the control unit controls the heating circuit to stop heating.

Optionally, the control unit monitors the temperature of the heating tube. When the temperature of the heating tube is greater than the first temperature threshold, the control unit controls the heating circuit to stop heating; and when the temperature of the heating tube is less than the second temperature threshold, the control unit controls the heating circuit to start heating, where the first temperature threshold is greater than the second temperature threshold.

Optionally, the first temperature threshold and the second temperature threshold are adjustable.

Optionally, the control unit monitors the temperature of the heating pad. When the temperature of the heating pad is greater than the third temperature threshold, the control unit controls the heating circuit to stop heating; and when the temperature of the heating pad is less than the fourth temperature threshold, the control unit controls the heating circuit to start heating, where the third temperature threshold is greater than the fourth temperature threshold.

Optionally, the third temperature threshold and the fourth temperature threshold are adjustable.

Optionally, the starting and stopping heating of the system are controlled according to time settings.

Optionally, the rechargeable battery is a plurality of batteries connected in series or parallel, or adopts a ring-shaped battery with a built-in empty space in the center.

Optionally, the display unit includes a first light-emitting diode (LED) for indicating the working state of the needle warming system.

Optionally, if the first LED is on, it indicates that the needle warming system is in the working state and the battery level is above a preset threshold; if the first LED flashes, it means that the needle warming system is in the working state, but the battery level is below a preset threshold; if the first LED is off, it indicates that the needle warming system stops working.

Optionally, the display unit includes a second LED for indicating the charging status, including whether the battery is fully charged, or is still charging.

Optionally, the needle warming system may further include a wireless charging receiving unit for charging the rechargeable battery or the rechargeable battery pack.

Optionally, the wireless charging station has multiple wireless charging transmitters.

The needle warming system of the present invention has one or more of the following benefits: (1) The needle warming system of the embodiment of the present invention uses a wirelessly rechargeable battery, thereby avoiding an external power supply and getting ride of wiring obstacles; (2) The creation of empty space from the ring-shaped rechargeable batteries, wireless charging receiving unit, heating pads, medicine pad, etc., allows the acupuncture needles to conveniently enter the heating tube and get heated, and the entire needle warming system is compact, portable and convenient to use; (3) The use of a conical guiding tube allows the acupuncture needle get into the heating tube easily; (4) The control unit can control the temperature of the heating tube and/or heating pad to adapt the needs of different users with different health conditions, thus enhance the comfort, experience and efficacy for different users; (5) The preferred, optional and personalized double-sided adhesive medicine pads can enhance the efficacy of needle warming acupuncture; (6) The configuration of a wireless charging station with multiple wireless charging transmitter can simultaneously charge multiple needle warming system; and (7) The needle warming system is equipped with a display unit for the displaying of working status, power status, charging status and temperature control status, thus the operator can clearly know the above status.

### BRIEF DESCRIPTION OF THE DRAWINGS

FIG. 1A shows a schematic diagram of the components in a needle warming system according to an embodiment of the present invention. FIG. 1B is a schematic assembly diagram of a needle warming system of this embodiment. FIG. 1C a longitudinal cross-sectional view of the needle warming system of this embodiment, and FIG. 1D is a product view of a needle warming system according of this embodiment.

FIG. 2 is a temperature control line for the needle warming system to control the heating tube according to an embodiment of the present invention.

FIG. 3 is a schematic diagram of a double-sided adhesive medicine pad according to an embodiment of the present invention.

FIG. 4 is a schematic diagram of a wireless charging station according to an embodiment of the present invention.

In above figures, the number labels are: 110 heating tube; 112, 145 thermal insulation material; 115, 135 temperature sensor; 120 control circuit board; 125 ring-shaped battery; 130 wireless charging receiving unit; 140 heating pad; 160 external case for the needle warming system; 170 system status indicator; 180 on/off button; 185 selection button for first, second, third or fourth temperature adjustment; 186, 187 temperature adjustment buttons, one to increase temperature, one to decrease temperature; 190 charging status indicator; 220 medicine pad; 210, 230 adhesive layers attached to two sides of the medicine pad; 310 power cord; 320 charging station; 330 charging locations; 340 charging transmitters.

### DETAILED DESCRIPTION

The following describes the embodiments of the present invention in detail with reference to the accompanying drawings.

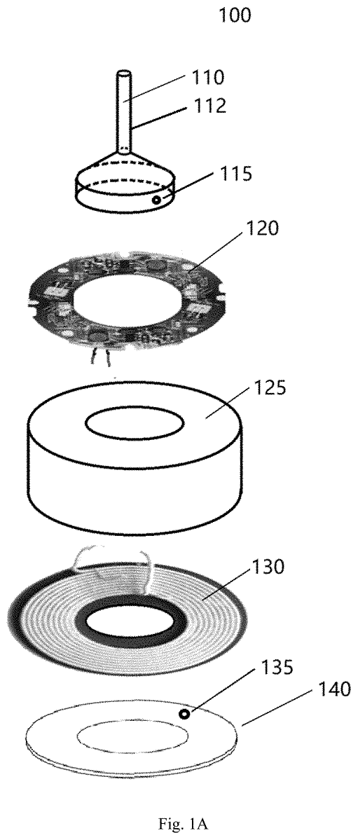

FIG. 1A shows a schematic diagram of the components in a needle warming system 100 according to an embodiment of the present invention. FIG. 1B is a schematic assembly diagram of a needle warming system of this embodiment. FIG. 1C a longitudinal cross-sectional view of the needle warming system of this embodiment, and FIG. 1D is a product view of a needle warming system according of this embodiment.

As shown in FIGS. 1A, 1B, 1C, and 1D, the needle warming system as an example may include: a heating tube 110, a ring-shaped battery 125 (rechargeable battery or rechargeable battery pack), and a control circuit board 120 (to control temperature, display and charging), display components 170, 188, 190, system on/off button 180, temperature control buttons 185, 186, 187.

The heating tube 110 is used to warm up the acupuncture needle by sheathing on the acupuncture needle handle. The heating tube may be, for example, MCH ceramic heating element, PTC ceramic heating element, polyimide metal heating film, carbon fiber heating material, or graphene heating material. The top of the heating tube 110 can be open to allow the needle coming through if long needles are used. When the acupuncture needle is relatively short, the acupuncture needle may not extend out of the opening of the heating tube. If the heating tube is manufactured long enough to accommodate all kinds of acupuncture needles available on the market, the top of the heating tube 110 can also be closed.

The ring-shaped battery 125 is a rechargeable battery or a rechargeable battery pack, and is used to supply power to the heating tube and the heating pad 140, if available. The rechargeable battery is charged through the wireless charging receiving unit in a wireless charging mode.

In another word, the rechargeable battery or rechargeable battery pack adopts a ring-shaped structure with empty space in the center, so that the needle warming system can be placed over and sheath the acupuncture needle in the heating tube. With this design, the adoption of the rechargeable battery and the wireless charging receiving unit does not affect the needle warming function from the heating tube.

In addition, the embodiment of the present invention uses a rechargeable battery as a power source for heating, thereby eliminating the wire of an external power source in the conventional technology. In this way, when multiple needle warming systems are used, there will not be many wires tangled together.

As an example, the rechargeable battery can be formed by connecting a plurality of batteries in series or in parallel in a ring shape, or can be molded directly into a ring shape. The empty space in the center of the ring-shaped battery allows the needle going through and resting in the heating tube to get heated.

In one example, a conical guiding tube 114 is formed at the bottom of the heating tube. When the needle warming system is put over the acupuncture needle, the acupuncture needle can be guided into the heating tube easily.

The display unit is used to display the system working status, battery level, charging status and temperature control status of the needle warming system.

In the example shown in FIG. 1D, the display unit includes a light emitting diode 170, serving as a working status indicator. An example of an implementation method is: when the light 170 is on, it indicates that the needle warming system is in working state and the battery level is above a preset threshold; when the light 170 is flashing, it indicates that the needle warming system is in working state, but the battery level is below a preset threshold; when the light 170 is off, it indicates that the needle warming system is off.

In the example shown in FIG. 1D, the display unit includes a light emitting diode 190, which serves as a battery charging status indicator. An example of an implementation method is: when the light 190 is off, it indicates that the battery is not charging; when the light 190 is red, it indicates that the battery is charging; when the light 190 is green, it indicates that the battery is fully charged.

The control unit 120, for example, includes a control circuit board and software running thereon, for controlling the needle warming system functions and parameters, such as the heating tube/heating pad temperature, the charging of the rechargeable battery, and the display of the display unit. The control circuit board can have a central processing unit such as a microprocessor and a memory integrated. The above control unit 120 serves as a temperature control unit, a charging control unit, and a display control unit together. However, the control unit 120 can also have separate units for temperature control, battery charging control, and display control.

The control unit 120 can include wireless communication components to receive remote controls from external system, to control the on/off of the system or to control the temperature of the heating tube, heating pad. The use of remote-control mode can make use of big data and artificial intelligence to prescribe better warm needle treatment and achieve better outcome.

The control unit 120 can control the temperature of the heating tube to ensure that the temperature does not exceed a safety limit.

In one example, the needle warming system further includes a normally closed temperature control switch. When the temperature of the heating tube exceeds a certain threshold, the control unit 120 controls the normally closed temperature control switch to automatically cut off the heating circuit.

In one example, the control unit 120 measures the temperature of the heating tube or the acupuncture needle through a temperature measuring element, and when the temperature of the heating tube/acupuncture needle exceeds a preset threshold, controls the heating circuit to stop heating.

In one example, the control unit 120 monitors the temperature of the heating tube, when the temperature of the heating tube is greater than a first temperature, controls the electrical circuit to stop heating; and when the temperature of the heating tube is less than a second temperature, controls the electrical circuit to start heating, wherein the first temperature is greater than the second temperature.

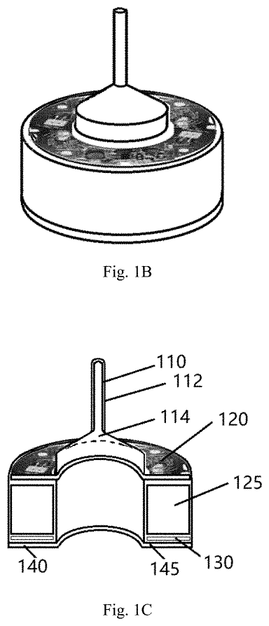

FIG. 2 shows a temperature line as an example of temperature control of a heating tube. Before the needle warming system is turned on, the room temperature is C0. After the system is turned on, the heating tube begins to get heated until the temperature reaches the preset temperature C2. Then the needle warming system stops to heat the heating tube, allowing it to naturally cool down to temperature C1. The cycle continues until the system is turned off.

In one example, the needle warming system further includes a heating pad for directly heating the human skin. Preferably the heating pad is located at the bottom of the needle warming system and has a flat ring shape. During the acupuncture process, if the needle warming system is dedicated for the user, the heating pad can directly contact the human skin to warm up the human skin. The heating pad of a non-dedicated system can heat up the human skin through a disposable double-sided adhesive tape, which can help to avoid possible cross infection between different patients. The heating pad of a non-dedicated system can also heat up the skin through a double-sided adhesive medicine pad, which can not only avoid the cross infection between patients, but also enhance the treatment outcome through the penetration of medicine through the skin from the medicine pad.

The control unit 120 may similarly control the heating pad temperature: starting from room temperature, the heating pad is heated up to a third temperature, followed by naturally cooling down to a fourth temperature. In detail, when the temperature of the heating pad reaches the third temperature, the control unit 120 controls to cut off the heating circuit; and when the temperature of the heating pad is less than the fourth temperature threshold, the control unit controls to switch on the heating circuit, wherein the third temperature threshold is greater than the fourth temperature threshold. Then the cycle continues until the system is turned off. The temperature change during the treatment procedure is same as in FIG. 2, except that C1 and C2, can be set to different values, and preferably, the values can be set dynamically through big data analysis and artificial intelligence.

The temperature of the heating tube and the heating pad can be controlled in other ways. For example, they can be simply heated for a predetermined time duration, for example one minute, and then the heating can be stopped for another predetermined time duration, for example one minute again. Then the cycle can be repeated.

The above-mentioned first temperature, second temperature, third temperature, and fourth temperature can be changed manually by buttons 185, 186, 187 according to the patient's condition and the acupuncture site. The button 185 selects the first, second, third, or fourth temperature for control, 186 is used to increase the temperature, and 187 is used to decrease the temperature.

In one example, the needle warming system further includes a double-sided adhesive medicine pad. One adhesive layer adheres to the needle warming system, and specifically on the bottom of the needle warming system. The other adhesive layer adheres to the user's skin.

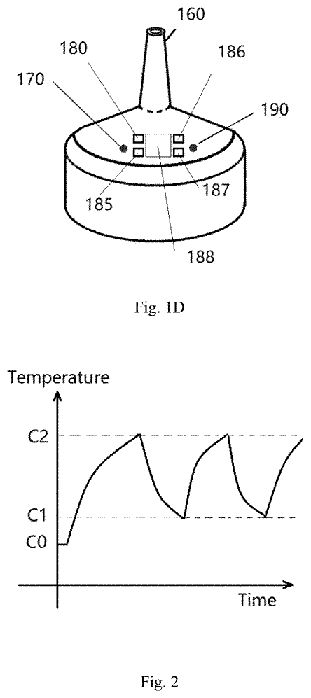

FIG. 3 shows a schematic diagram of a disposable double-sided adhesive medicine pad. Adhesive layers 210 and 230 are attached to two sides of the medicine pad 220. One adhesive layer is used to connect the medicine pad to the bottom of the needle warming system, and the other adhesive layer is used to connect the medicine pad to the skin. There are two benefits of such a double-sided adhesive medicine pad: 1) The disposable double-sided adhesive medicine pad prevents cross infections between patients, especially for some skin diseases; 2) The medicine pad can enhance the treatment outcome of the warm needle technique. The adhesive layer between the medicine pad and the needle warming system does not need to be permeable to the medicine. The adhesive layer between the medicine pad and the skin needs to be permeable, which allows the medicine in the medicine pad to penetrate through the adhesive layer into the skin. The medicine infiltrated in the medicine pad can be of different property, such as promoting blood circulation, removing dampness, warming yang or nourish yin.

In one example, considering the safety problem, energy saving problem, and system control convenience, heat insulation layers 112 and 114 are integrated to the system. The insulation layers contain the heat near the heating tube and heating pad to minimize the heat loss and save the battery, to prevent the overheating of the electronic components and the rechargeable battery, to prevent burns of skins of the patient or the nearby people, and to prevent the heat conduction between the heating tube and heating pad for better individual temperature control.

In the example shown in FIGS. 1A-1D, the needle warming system further includes: a wireless charging receiving unit 130, which is ring-shaped and assembled below the rechargeable battery. When the needle warming system is placed on the charging station, the wireless charging receiving unit charges the rechargeable battery with electricity generated by the wireless charging transmitters in the charging station.

In one example, the needle warming system is also accompanied with a wireless charging transmitter or a group of transmitters, which is/are ring-shaped and used to charge a rechargeable battery or a rechargeable battery pack through the wireless charging receiving unit.

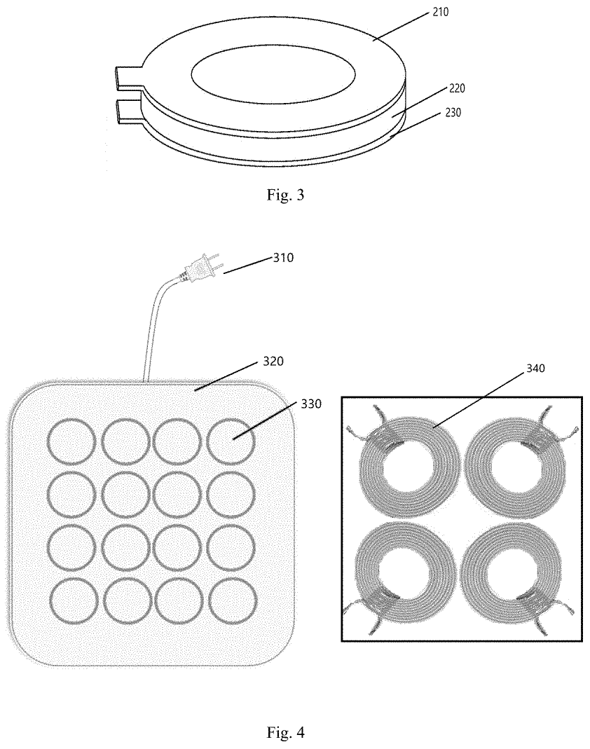

FIG. 4 shows a wireless charging station 320 with a power cord 310. The wireless charging station has four wireless charging transmitters 340. The circles 330 indicatively show the locations the needle warming system can be put on to get charged.

The needle warming system of the present disclosure has one or more of the following benefits: (1) The needle warming system of the present invention uses a wireless rechargeable battery, thereby avoiding external power supply and wires; (2) The creation of empty space in the center through the use of ring-shaped rechargeable batteries, heating pads, medicine pads, etc. facilitates the entry of acupuncture needles into the heating tube to get heated up. The entire needle warming system is compact, portable and easy to use; (3) The design of a conical guide tube below the heating tube in a preferred embodiment can guide the acupuncture needle into the heating tube easily; (4) The control unit can control the temperature of the heating tube and/or heating pad dynamically and adaptively according to the needs of different users, enhancing the comfort, experience, and treatment effect of different users; (5) The preferred and optional double-sided adhesive medicine pads can enhance the efficacy of warm needle technique and avoiding cross infection between different users; (6) The configuration of a multiple wireless charging transmitters makes it possible to charge multiple needle warming systems simultaneously; and (7) The needle warming system is equipped with a display unit to display working status, battery level status, charging status and temperature control status, so that the operator can clearly know the above status.

The embodiments of the present invention have been described above. The above description is exemplary, not exhaustive, and is not limited to the disclosed embodiments. Many modifications and variations will be apparent to those of ordinary skill in the art without departing from the scope and spirit of the illustrated embodiments. Therefore, the protection scope of the present invention should be subject to the protection scope of the claims.

1. A needle warming system, including:
a heating tube, used to heat up acupuncture needles;
a ring-shaped rechargeable battery to provide power to the heating tube, with the empty space in the center allowing the acupuncture needle to get through into the heating tube, wherein the ring-shaped rechargeable battery being charged by wireless charging transmitters in a wireless mode;
a display unit to display the system's working status and battery charging status;
a control unit used to control the heating tube's temperature, the rechargeable battery's charging, and the display unit's displaying.

2. The needle warming system according to claim 1, further comprises:
a heating pad located at the bottom of the needle warming system to directly warm the human skin.

3. The needle warming system according to claim 1, further comprises:
double-sided adhesive pad, one side being used to stick to the needle warming system, and the other side being used to stick to the user.

4. The needle warming system according to claim 3, wherein the double-sided adhesive pad comprises:
a first adhesive layer, a medicine pad and a second adhesive layer, with the first adhesive layer attaching the medicine pad to the needle warming system, and the second adhesive layer attaching the medicine pad to the user's skin, wherein the adhesive layer between the medicine pad and the skin is permeable for the medicine.

5. The needle warming system according to claim 1, further comprises:
heat insulation layers assembled around the heating tube and above the heating pad, which contain heat near the heating tube and heating pad, preventing the direct contact of the heating tube with the skin of the user or other nearly persons to cause burns, preventing the overheating of electronic components and the battery, and reducing the heat conductance between the heating tube and heating pad for better separate temperature controlling of these two.

6. The needle warming system according to claim 1, further comprises:
a ring-shaped wireless charging receiving unit assembled below the rechargeable battery, which can be charged up from wireless charging transmitter upon placing on a charging station.

7. The needle warming system according to claim 1, wherein the heating tube is made of an MCH ceramic heating element, a PTC ceramic heating element, a polyimide metal heating film, a carbon fiber heating material or a graphene heating material.

8. The needle warming system according to claim 1, wherein one end of the heating tube pointing inwards connects with a conical guiding tube, which allows the acupuncture needle to sheath into the heating tube easily.

9. The needle warming system according to claim 1, further comprises a normally closed temperature control switch, which shuts off the heating circuit when the temperature of the heating tube exceeds a certain threshold.

10. The needle warming system according to claim 1, wherein the control unit further comprises a wireless communication unit, is able to receive remote controls from external system to control the needle warming system, controlling the turn-on/turn-off of the system and the temperature.

11. The needle warming system according to claim 1, wherein the control unit measures the temperature of the heating tube or the acupuncture needle through the temperature measuring element, and when the temperature of the heating tube exceeds a certain threshold, the control unit controls the heating circuit to stop heating.

12. The needle warming system according to claim 1, wherein the control unit measures the temperature of the heating tube or the acupuncture needle through a temperature measuring element, and when the temperature of the heating tube is greater than a first temperature, controls the heating circuit to stop heating; and when the temperature of the heating tube is less than a second temperature, controls the heating circuit to start heating, wherein the first temperature is greater than the second temperature.

13. The needle warming system according to claim 12, wherein the first temperature and the second temperature are adjustable manually or automatically.

14. The needle warming system according to claim 2, the control unit monitors the temperature of the heating pad, and when the temperature of the heating pad is greater than a third temperature, controls the heating circuit to stop heating; and when the temperature of the heating pad is less than a fourth temperature, controls the heating circuit to start heating, wherein the third temperature is greater than the fourth temperature.

15. The needle warming system according to claim 13, wherein the third temperature and the fourth temperature are adjustable.

16. The needle warming system according to claim 1, wherein the starting and stopping of the heating are controlled according to time settings.

17. The needle warming system according to claim 1, wherein the rechargeable battery is a plurality of batteries arranged in series or in parallel, or adopts a customer-designed ring-shaped battery.

18. The needle warming system according to claim 1, wherein the display unit comprises a first light emitting diode (LED) for indicating the working status of the needle warming system, with the light-on status of the LED indicating that the needle warming system is in a working status and the battery level is above a preset threshold; the flashing of the LED indicating that the needle warming system is in a working status, but the batter level is below a preset threshold; the light-off status of the LED indicating that the needle warming system is off.

19. The needle warming system according to claim 1, the display unit includes a second LED for indicating the charging status, including whether the system is fully charged or is charging.

20. The needle warming system according to claim 1, further comprising a wireless charging station with one or more wireless charging transmitters, to charge the rechargeable battery or the rechargeable battery pack.
